(19) United States
(12) Patent Application Publication
(10) Pub. No.: US 2010/0261618 A1
(43) Pub. Date: Oct. 14, 2010

(54) Identification of genes implicated in the virulence of streptococcus agalactiae

(76) Inventors: Sonia Escaich, Paris (FR); Francois Moreau, Orsay (FR)

Correspondence Address: NIXON & VANDERHYE, PC, 901 NORTH GLEBE ROAD, 11TH FLOOR, ARLINGTON, VA, 22203, US

(21) Appl. No.: 12/308,248

(22) PCT Filed: Jun. 19, 2007
(86) PCT No.: PCT/IB2007/002781

§ 371 Date: Jun. 18, 2010

Related U.S. Application Data
(60) Provisional application No. 60/814,535, filed on Jun. 19, 2006.

(51) Int. Cl. C40B 30/06; C12N 15/74; C07H 21/04; C07K 14/315; C12Q 1/02

(52) U.S. Cl. 506/10; 435/471; 536/23.1; 530/350; 435/29

(57) ABSTRACT

Group B streptococcus is an important cause of maternal and neonatal morbidity and mortality in many part of the world. The invention is a method of identification of novel targets for inhibitors preventing septicemic dissemination of Streptococcus agalactiae, a model of Gram positive bacteria, in order to treat bacterial infections using these virulence determinant.

### BACKGROUND

Group B Streptococcus (Streptococcus agalactiae or GBS) is a Gram positive bacteria and is a widespread commensal of the human genital and intestinal tract. GBS has emerged as an important cause of human disease and is now the most common cause of life-threatening invasive bacterial infections (septicaemia, pneumonia, and meningitis) during the neonatal period (Gibbs et al. 2004. Obs Gyn 104:1062-76), and a major cause of mortality in immunocompromised adults (Farley. 2001. Clin Inf Dis. 33:556-61). Newborns infections result either from the passage of the bacterium through the placental membrane or by the inspiration of the bacterium of infected vaginal flora during delivering. GBS adhere to a variety of human cells including vaginal epithelium, placental membranes, respiratory tract epithelium and blood-brain barrier endothelium.

On the nine GBS identified antigenically distinct serotypes based on their capsular polysaccharide structure. The types Ia, Ib, II, III, and V are responsible for the majority of invasive human GBS disease. Serotype III GBS is particularly important because it causes a significant percentage of early onset disease (i.e. infection occurring within the first week of life) and the majority of late-onset disease (i.e. infection occurring after the first week of life). Overall, the capsular serotype III is responsible for most cases (80%) of neonate GBS meningitis.

Bacteria have developed specific mechanisms to evade complement, an important arm of the innate immune system and effector in the adaptative immune system. However, complement is not sufficient to prevent GBS systemic invasion.

Cationic antimicrobial peptides (CAP) play a fundamental role in innate immune defenses, both through direct antimicrobial activity and through immunomodulatory effects. The CAP dominating targets are bacterial membranes and the killing reaction must be faster than the growth rate of the bacteria. Clinical cases show that deficiencies in these peptides give severe symptoms. The activities of the cationic peptides against S. agalactiae increase as the bacterial electropositive charge surface decreases.

The growing prevalence of antibiotic-resistant bacteria has increased the complexity of anti-infective therapies being administer in hospitals. While the phenomenon of resistance is not new, it has become of increasing concern as more and more antibiotics are rendered ineffective. Given this situation, there has been an urgent need to develop new bactericidal agents which target resistant Gram-positive pathogens and particularly in the GBS infection.

The availability of the complete genome sequence of GBS open the way of identification of genes implicated in systemic dissemination of bacteria.

The major described virulence factors of GBS are the polysaccharide capsule, the lipotechoic acid, the hemolysin, the C5-peptidase, the superoxide dismutase and the protease CspA (Lindahl et al, 2005, Clinical Mic review). The analysis of 1600 mutants of a type Ia strain by signature-tagged mutagenesis (STM) in a neonatal rat model by Jones et al. (Jones et al, 2000, Mol Mic 37:1444-1455) had identified novel genes implicated in GBS virulence. Most of the genes identified affected transport, regulation and adherence functions, highlighting their role in GBS pathogenicity. However, because of the technical restraints and limitations of STM it is likely that this work is not exhaustive and thus, that many other genes implicated in GBS virulence are yet unknown.

The aim of the invention is to provide new genes implicated in the GBS virulence. For this purpose, the inventors report the construction, by STM method and screening for cell wall defects, an insertion mutant library of a serotype III S. agalactiae. Colistin, an antimicrobial peptide and Novobiocin (an antibiotic) screening leads to the identification of 97 genes. 27 mutants were tested in an animal model and 13 were less virulent than the wild type strain. 8 new genes were identified that are important for GBS virulence. These genes are new target for antimicrobial drugs.

The invention thus relates to a method for the insertion mutagenesis of GBS comprising the use of vector pTCV-Tase (see Material and Methods).

It also relates to the insertion mutant of genes gbs 0052 (SEQ ID N° 1), gbs 0100 (SEQ ID N° 2), gbs 0307 (SEQ ID N° 3), gbs 0582 (SEQ ID N° 4), gbs 0653 (SEQ ID N° 5), gbs 0683 (SEQ ID N° 6), gbs 1787 (SEQ ID N° 7), gbs 2100 (SEQ ID N° 8).

The invention also relates to an in vitro Screening Method of the mutants library comprising using colistine as a mimic of innate immunity components that are the antibacterial cationic peptides and using novobiocine to detect mutant with defect in outer membrane permeability. The methods of Combination of the in vitro screening results with in vivo effect of mutations, to identify target proteins having en essential function for in vivo virulence are also part of the invention.

The invention also relates to the proteins sequences of these targets in GBS as useful to find drugs preventing bacterial dissemination in the host and called antivirulence targets.

Another object of the invention relate to the proteins sequence in GBS and homologous sequences in gram positive bacteria with at least 22% identify on the full length seq and 25% identity in a 100 continuous amino acid sequence. In particular homologous proteins present in Streptococcus Pneumoniae (SPN), to find drugs for treating Gram positive infections.

The biochemical assays developed to screen for small molecules inhibitors and described hereinafter also enter into the scope of the invention.

The invention thus relates to a biochemical assay for screening inhibitors for GBS characterized in that they are based either on luminescent ATP or fluorescent ADP detection.

The biochemical assay based on luminescent detection comprises:
adding a substrate mixture comprising, GBS, myelin basic protein and ATP to an assay buffer preincubated with DMSO or analog or inhibitor dissolved in DMSO or analog,
incubating at room temperature,
adding a revelation mixture, and
measuring luminescence.

The Biochemical Assay Based on Fluorescent Detection Comprises:

adding a substrate mixture comprising GBS, myelin basic protein; ATP, pyruvate and NaDH,
measuring fluorescence intensity of NaDH (λₑₓ=360 nm, λₑₘ=520 nm),
deriving the inhibition % from fitted initial velocities.

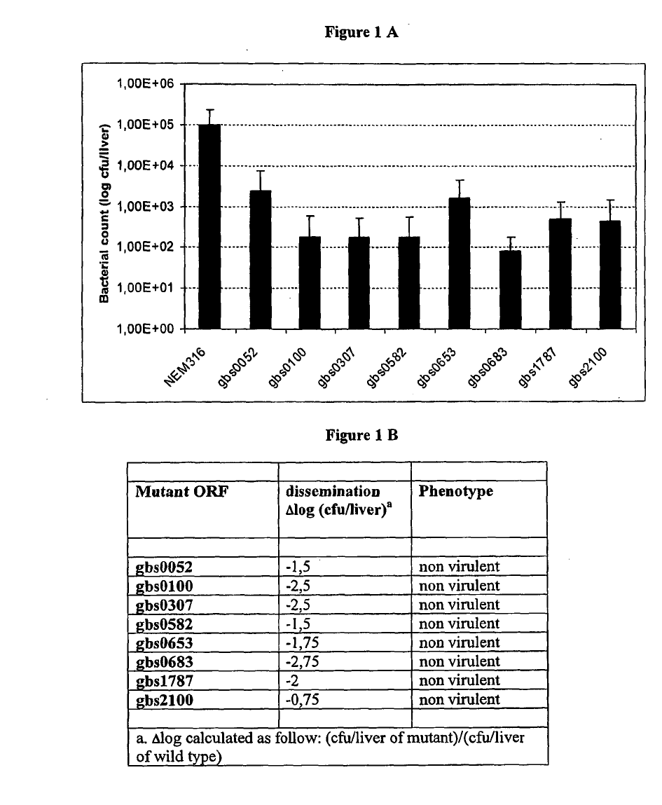

Other characteristics and advantages of the invention are given in the following examples wherein it is referred to FIGS. 1 to 4, with:

FIG. 1 representing: (A) bacterial counts for GBS and (B) results concerning Mutant ORFs,

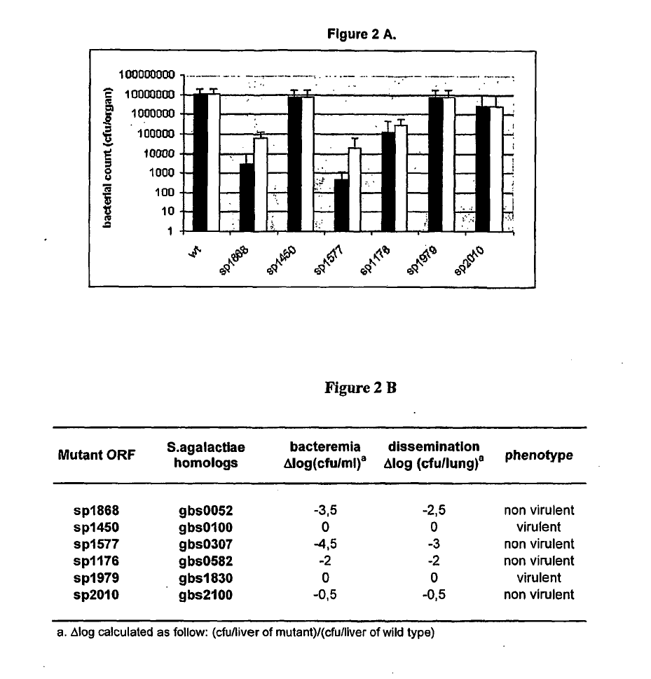

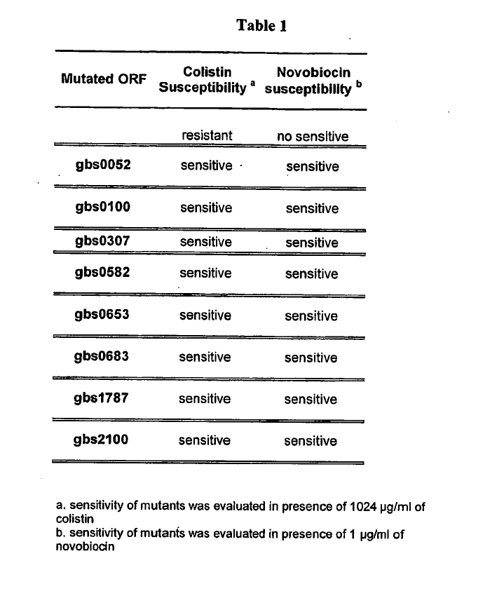

FIG. 2: (A) bacterial counts for SP and (B) results concerning Mutant ORFs,

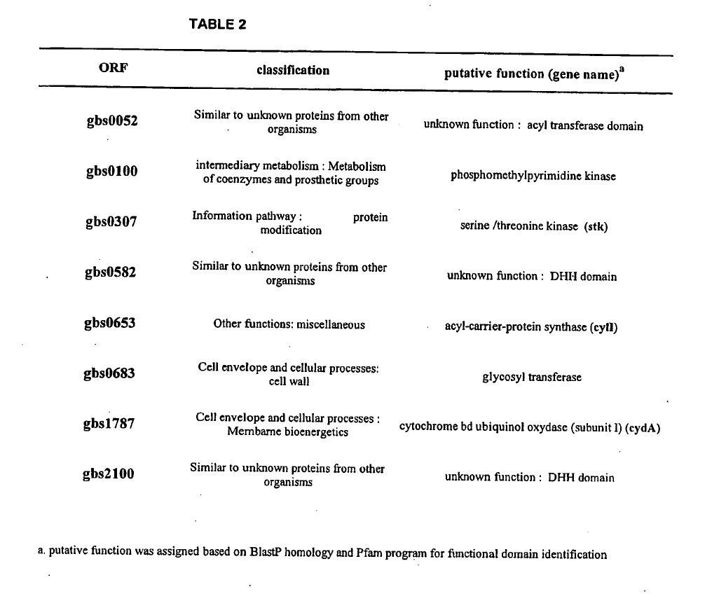

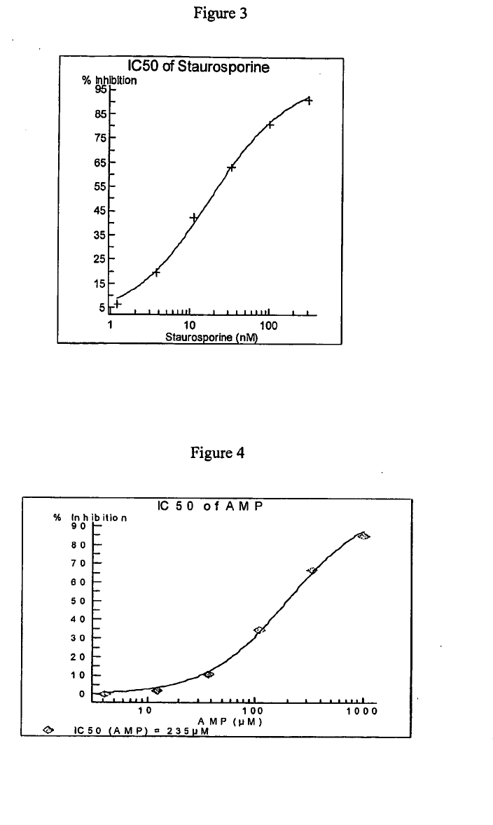

FIG. 3: IC₅₀ of Staurosporine

FIG. 4: IC₅₀ of AMP

### MATERIALS AND METHODS

Bacterial strains, media and growth conditions. E. coli strain TOP10 (Invitrogen) was used for DNA cloning and plasmid propagation. E. coli were grown on liquid or solid Luria-Bertani (LB) medium at 37° C.

Streptococcus agalactiae NEM316, whose sequence has been determined by the Pasteur Institut (Glaser et al, 2002. Mol. Mic. 45:1499-513), is responsible for a fatal septicaemia and is of to the capsular serotype III (Gaillot et al 1997 gene 204:213-218). GBS strains were grown in Todd-Hewitt (TH) broth or agar (Difco Laboratories, Detroit, Mich.) at 37° C., unless otherwise specified.

Because Streptococcus pneumoniae R6, whose genome has been sequenced (Hoskins et al, J. Bacteriol. 2001 October; 183(19):5709-17), is not virulent, for its virulent progenitor S. pneumoniae D39 was used, which is a clinical isolate obtained in 1916, that is commonly used in studies on the pathogenesis of pneumococcal infections. S. pneumoniae strains were grown in TH media unless otherwise stated at 37° C. in 5% CO₂.

For antibiotic selection of E. coli strains, kanamycin (Km) was used at 60 μg/ml and erythromycin (Em) at 150 μg/ml. To select strains derived from S. agalactiae NEM316 and S. pneumoniae D39, the Km concentration was 1000 μg/ml and Em at 10 μg/ml.

Genetic techniques and DNA manipulations. Genomic streptococcal DNA was isolated from overnight culture in TH supplemented with 0.6% glycine. Bacteria were harvested for 5 min at 5000×g then resuspended in 600 μl of cold PBS. Bacterial suspension was added to lysing Matrix B (QBiogen) and bacteria were mechanically disrupted using a Fast Prep instrument (Qbiogen). After centrifugation at 5000×g for 5 min, the supernatant was transferred to a fresh tube and DNA was extracted with the Wizard® Genomic DNA Purification Kit (Promega) according to the manufacturer's instructions.

Southern blot analysis was carried out as recommended (Sambrook, et al.). DNA sequences were performed by Genoscreen (Lille, France).

Plasmid DNA preparations were isolated using Wizard® Plus Minipreps DNA Purification System (Promega).

Construction of vector pTCV-Tase. Oligonucleotides Kana-5 out of SEQ ID N° 9 (5′-CCTATCACCTCAAATGGTTCGCTGGG-3′) and Kana-3 out of SEQ ID N° 10 (5′-CTGGGGATCAAGCCTGATTGGGAG-3′) were used to amplify the plasmid pTCV-erm (Poyart et al. 2001 J Bac 183: 6324-6334) without the gene aphA3. The PCR product was blunted, phosphorylated and then recircularised. The pair of oligonucleotides TaseF of SEQ ID N° 11 (5′-ATATCCATGGATGGAAAAAAAGGAATTTCGTG-3′) and TaseR of SEQ ID N° 12 (5′-AATCTGCAGTTATTATTCAACATAGTTCCCTTC-3′) was used to amplify a promoterless Himar1 transposase C9 gene from the DNA of the vector pET29C9 (Lampe et al. 1996 EMBO J. 15:5470-5479). After digestion with NcoI and PstI (sequence in bold), this amplicon was cloned in the multiple cloning site of pTCV-erm deleted for aphA-3. A 0.5 Kb EcoRI-NcoI DNA fragment containing the PaphA3 promoter (Poyart et al. 1997 FEMS Microbiology Letters 156:193-198) was inserted upstream of the transposase gene to give pTCV-Tase.

Electroporation of S. agalactiae. Bacteria were grown overnight at 37° C. in TH supplemented with 0.6% glycine. The culture was diluted to 1:10 into 500 ml TH with 0.6% glycine and allowed to grow until the optical density at 600 nm (OD₆₀₀) was between 0.3 and 0.5. The culture was harvested by centrifugation at 5000 rpm for 10 min, washed twice in 100 ml of cold sterile washing buffer (9 mM NaH₂PO₄, 1 mM MgCl₂, 0.5 M sucrose, pH7.4), and resuspended in 3 ml of washing buffer plus 10% glycerol, and frozen in aliquots or used directly.

For electroporation, 500 ng to 1 μg of plasmid DNA was added to 75 μl of the cell suspension on ice, and transferred to prechilled 2-mm electroporation cuvettes (BioRad Laboratories) and electroporated at 25 μF, 2500 V and 200 S2 with a Bio-Rad Gene Pulser apparatus. The suspension was diluted immediately into 1 ml of TH with 0.25 M sucrose and incubated for 3 h at 37° C. and then plated on TH agar plates containing the appropriate antibiotic.

Transformation of S. pneumoniae. Pneumococcal cells were transformed according to the protocol described by Echenique et al. Briefly, bacteria were grown to an OD₄₀₀ of 0.1 to 0.2 at 37° C. in CTM pH 7, and then frozen in 10% glycerol. Bacteria were thawed, centrifuged and resuspended in CTM pH 8.50 ng/ml. CSP was added and cells were incubated for 10 min at 37° C. The transforming DNA (0.01-0.05 μg/ml) was then added. Optimal DNA uptake was obtained by a 20 min incubation of the mixture at room temperature. The mixture was then diluted 1:10 in CAT medium and incubated at 37° C. for 2 h to allow chromosome segregation and phenotypic expression. Transformants were selected by plating in appropriate conditions and individual colonies were taken for analysis.

Generation of a signature-tagged S. agalactiae mutant library. Plasmid pTCV-Tase was electroprated in S. agalactiae NEM316 to give “Sa NEM-Tase”. This new low copy vector plasmid directs synthesis of the transposase in S. agalactiae and can be readily lost following subculture at 40° C. in the absence of antibiotic selective pressure. The strain “Sa NEM-Tase” was then electroporated with suicide plasmids containing Himar1 inverted repeats flanking a kanamycin cassette and one of the 80-bp oligonucleotide signature tags kindly given by V. Pelicic (Geoffroy et al. 2003 Genome research 13:391-398). Bacteria were allowed to recover 3 h in TH with 0.25M sucrose at 37° C. and then plated onto TH plates containing kanamycin. Thus, only bacteria that have undergone in vivo transposition events were selected.

Determination of Himar1 insertion sites. The identification of genomic DNA sequences flanking the inserted transposons was done by ligation-mediated PCR (LMPCR) (Prod'hom et al. 1998) as described (Pelicic et al. 2000). Briefly, genomic DNA was digested by Sau3Al, which generate short DNA fragments. The linkers were formed by annealing LMP1 of SEQ ID N° 13 (5′-TAGCTTATTCCTCCAAGGCACGAGC-3′) with LMP21 of SEQ ID N° 14 (5′-GATCGCTCGTGCCTT-3′); the underlined sequences correspond to complementary sequences in the primers, whereas sequences complementary to cohesive ends generated by Sau3Al are in bold. Linkers were ligated to digested DNA, and then the insertion sites were amplified with AmpliTaq Gold DNA polymerase (PE Applied Biosystem) using LMP1 and ISR of SEQ ID N° 15 (5′-CGCTCTTGAAGGGAACTATGTTGA-3′) or ISL of SEQ ID N° 16 (5′-AATCATTTGAAGGTTGGTACTATA-3′), outward primer internal to the mini-transposon. PCR products were gel purified and directly sequenced using ISL or ISL as primer. Sequence homology searches were performed using BLASTN against the streptococcal sequences present in the databases.

Screening for sensitivity to cationic peptide colistin and cell wall defect of the mutant library. The S. agalactiae mutants were grown TH-Kn in 96-wells microplates over-night. Bacteria were then diluted to 1:1000 and each dilution was dispatched into two microplates. In one microplate 50 μl of TH was added, whereas 50 μl of 512 μg/ml colistin (Sigma) or 2 μg/ml novobiocin (Sigma) was added in the other thus obtaining final concentrations of 256 μg/ml and 1 μg/ml, respectively. Microplates were incubated overnight at 37° C. OD₆₀₀ of microplates was read on a Multiskan Ex apparatus (Thermo).

Construction of gene knockout mutants. To construct S. agalactiae deletion mutants, the coding sequence of the gene of interest was replaced with a promoterless and terminaterless kanamycin resistance cassette aphA-3 (Trieu-Cuot and Courvalin, 1983). This was done by ligating successively, after digestion with the appropriate enzymes, the three amplicons into pG+host5 and introducing the resulting recombinant vectors by electroporation into NEM316. The double cross-over events leading to the expected gene replacements were obtained and verified as described (Biswas et al. 1993. Journal of bacteriol).

S. pneumoniae deletion mutants were achieved by transforming wild type strain with pUC19 plasmid in which was cloned a PCR product containing the aphA-3 cassette flanked by the 5′ and 3′ 90-pb of the gene of interest.

Animal model. Mouse virulence studies were performed using 3-week-old female BALB/c@Rj mice (Janvier laboratories).

GBS NEM316 and derivative mutants were grown to exponential phase in broth culture. 200 μl of a bacterial suspension of 3.10⁷ CFU/ml were administered by intravenous injection to groups of eight mice (6.10⁶ CFU/mouse). Exact inoculum numbers were determined by plating 10-fold dilutions of the suspension on TH agar plates immediately after inoculation. At 44 hours post-infection, mice were sacrificed by cervical dislocation. The abdominal cavities of the mice were aseptically opened, and the livers were removed. Livers were homogenized with a tissue homogenizer (Heidolph) in 1 ml of sterile 0.9% NaCl, and 10-fold serial dilutions were plated on TH agar plates to determine bacterial loads.

Pneumococcus infection was carried out in a similar manner except that bacterial inocula contained 3.10² CFU/mouse in a volume of 100 μl. Virulence analysis was based on recovery of CFU in lungs and blood at 44 h postinfection.

All animal experiments were carried out in accordance with institutional guidelines.

### DESCRIPTION

### Construction of the Signature-Tagged S. Agalactiae Library and Stability

DNA sequence coding for transposase was cloned in a gram-positive replicative plasmid under weak promoter (pTCV-Tase) allowing expression of transposase once the plasmid was introduced into S. agalactiae NEM316 strain. As shown in FIG. 1, transposase-containing plasmid included a thermo sensitive replication origin that permitted an efficient lost of the plasmid at non-permissive temperature. A library of signature-tagged insertion mutants of S. agalactiae was constructed as described, electroporating a suicide vector with the tagged transposon into a strain expressing the transposase. Forty-eight tagged transposons, each labelled with a different signature tag, were used to produce the library. Ninety-six randomly picked mutants of each tag were organised on microplates that were immediately frozen at −80° C. in 20% glycerol. Thus a library of 4608 viable mutants was obtained.

HindIII-digested DNA of 15 randomly picked transformants obtained from a single electroporation experiment was analysed by Southern blotting using the aphA-3 cassette as a probe. A single hybridizing fragment was detected for each mutant indicating that a unique transposition event has occurred for a given mutant. Moreover, the size of the hybridising DNA fragments was different in each case, from 2 kb up to 10 kb in size (FIG. 1), suggesting a random insertion of the transposon into the chromosome of GBS. Similar results could be obtained with by Southern analysis of EcoRI-digested chromosomal DNA.

During the construction of the library, difficulties were encountered in eliminating efficiently transposase-expressing plasmid. To study the stability of mutations in S. agalactiae mutant keeping transposase-containing plasmid, three strains were randomly chosen and their site of transposon insertion was sequenced after the strains had been subcultured every day in fresh medium for 13 days. For each of them, the site of insertion was identical at J0, J3, J8 and J13. This result indicates that the insertion in the chromosome is stable despite the presence of the plasmid pTCV-Tase, which expresses the transposase. This last point suggests either that the expression of the transposase by pTCV-Tase is not sufficient to lead to a second event of transposition in those conditions or that the conditions tested do not allow further transposition.

Screen for Genes Implicated in S. Agalactiae Resistance to Cationic Peptides.

In common with other polymixins, colistin is rapidly bactericidal and exerts its effect by acting as a cationic detergent, causing disruption of the integrity of the bacterial cell membrane, with leakage of intracellular contents and cell death (Catchpole C. R et al, 1997, j. antimic. chem.). Colistin minnics the effets of antimicrobial cationic peptides of innate immunity.

Thus, mutants showing an increased sensitivity to colistin might have a transposon insertion in a gene implicated in the structure of the envelope. In total, 41 mutant strains were identified as being more sensitive to colistin since they were unable to grow at a concentration of 256 μg/ml in opposition to the wild type strain (CMI≦1024 μg/ml).

Novobiocin was chosen as second antibiotic to select mutants presenting defects in their outer membrane. This hydrophobic antibiotic need to pass through the cell wall of the bacteria in order to reach its target: the bacterial type II topoisomerases DNA gyrase and topoisomerase IV, thus alteration of the cell-wall could lead to an increase of sensitivity to novobiocin, as well as mutants of the DNA metabolism. Screening for sensitivity to novobiocin has been previously used to select cell wall defective mutant in gram positive bacteria (Lui et al. 1999 PNAS). In this manner, 155 mutant clones were identified as being more sensitive to novobiocin, 46 of these were also more sensitive to colistin.

Altogether, 196 mutants were revealed sensitive to colistin and/or Novobiocin in the tested in vitro condition. As most of the mutants defective for growth with 256 μg/ml of colistin were also more sensitive to novobiocin, the MIC of colistin on all the mutants detected by the colistin and novobiocin screens were determined in order to verify that any colistin sensitive mutant had not been missed with the screen. Results are shown in Table 2 and indicate that none of the mutants revealed only by the novobiocin screen were sensitive to colistin.

Mapping of Transposon Insertion Sites of Colistin and/or Novobiocin Sensitive Clones.

The insertions sites of the transposon in the colistin and/or novobiocin sensitive clones were determined by LM-PCR and sequencing (see Materials and Methods). Using the genome sequence database obtained from Pasteur, mutated ORFs were identified. Thus, it was observed that the 170 mutant strains presenting a defect for growth in presence of 256 μg/ml colistin and/or 1 μg/ml novobiocin correspond to 89 ORFs (Table 2).

Some genes had undergone several mutations, but the sequencing revealed that the transposition sites were different, in the large majority of cases, suggesting that there were no hot spots for transposon insertion. Where insertion sites were identical, the clones were probably siblings as they had, in each case, come from the same electroporation event.

According to the functional classification of gene established after genome sequencing of NEM316 (Glaser et al, 2002. Mol. Mic. 45:1499-513), one third (56) of the mutants revealed by the screen were in genes classified as implicated in the cell envelope and cellular processes. One-sixth (31) corresponded to mutants of genes implicated in intermediary metabolism and almost the same number (29) were mutants of genes important for information pathways, adaptation to atypical conditions or detoxification. Finally, one third (54) of the mutants had unknown functions. These results confirm that the screen was effective in finding genes implicated in the structure of the cell envelope.

Assessment of the Role of the Identified Mutants In Vivo.

It is likely that some of the bacterial genes revealed by the screens contribute to the GBS pathogenicity. To test this hypothesis, target genes revealed by sequencing were submitted to analysis based on significant detection of homologs in the genome of other relevant gram-positive pathogens. In addition, implication of these genes in the cell wall architecture was investigated using BlastP program. By this way, 27 mutants, sensitive to novobiocin and colistin, were selected among genes that were conserved among gram positive bacteria with a putative function in cell wall metabolism. These mutants were tested for virulence phenotype in an intravenous model of infection, as described in the Materials and Methods section. Results are shown in FIG. 3. Thirteen of the tested mutant strains showed a 0.5 to 3 log₁₀ decrease in CFU in the liver compared to the wild type strain. nine mutant strains behaved as did the wild type parental strain and only five of the mutant strains presented a higher significantly number of CFU than the wild type.

The identification of mutant with decrease liver dissemination suggests a role of these chromosomal loci in the virulence of GBS. To assess whether this association was strictly dependent of the mutated gene, a specific deletion was created in 10 genes by allelic replacement in the chromosome of GBS to generate isogenic mutant Mutants of GBS were injected to mice and bacterial clearance in the liver was measured. As shown in FIG. 4, all deletion mutants of exhibited a reduced bacterial count relative to the wild type. These results were comparable to those obtained with insertion mutant confirming specific linkage between mutated gene and hypo-virulent phenotype.

General growth defect causing attenuated virulence were ruled out by generating growth curve of individual deletion mutants grown in parallel with the wild type strain NEM316. The growth of all strains listed in FIG. 4 was found to be essentially identical to that of the parent strains NEM316, except for gbs1830, which exhibited a mild in vitro growth curve deficit.

Functional Analysis of Virulence-Associated Genes

According to the functional classification, which has been assigned during the sequencing of GBS, genes identified to be important for the virulence of GBS could be grouped in various classes (Table 2).

Gbs1787 showed significant homology to cydA of mycobacterium smegmatis. CydA encodes a subunit of cytochrome bd quinol oxidase. It is involved in energy transducing respiration in many prokaryote including E. coli (copper P A J. bact. 1990) and bacillus species (Winstedt et al. J. bact. 1998). In GBS, inactivation of cydA gene induced changes in growth characteristics (yamamoto et al.).

Gbs0683 was previously identified in GBS as iagA following an in vitro screen of a mutant library for loss of invasion phenotype to endothelial cell. IagA share homology to putative sugar transferase from other gram-positive bacteria. IagA function as a glycosyltransferase that catalyze the formation of DGIcDAG, a glycolipid that allow the anchoring of LTA to the bacterial cell wall (Doran et Al. JIC 2005).

Three genes were similar to unknown protein from other organism. However, some putative function might be inferred from protein sequence annotation. An acyl transferase domain was found on gbs0052 gene product. In streptococci, proteins with acyltransferase activity were involved in many biological processes including synthesis of peptidoglycan or capsular polysaccharide. Annotation of Gbs0582 and gbs2100 revealed the presence of a DHH motif. This domain composed of one aspartate and two histidine residues was associated with proteins of phosphodiesterase function including E. coli protein RecJ (Han E S Nucleic Acids Res. 2006).

Gbs0307 gene product belongs to the eukaryotic-type serine/threonine kinase family. Serine/threonine kinases were present in various gram-positive bacteria including pknB of mycobacterium tuberculosis (Av-Gay et al. AIA 1999). Gbs307 has been characterized as stk1 in GBS, a kinase that phosphorylate various substrates on serine and threonine residues (Jin H et al. J Mol. Biol. 2006; Rajagopal L et al. J Biol. Chem. 2003; Rajagopal L et al. Mol. Microbiol. 2005). Inactivation of stk1 impaired bacterial growth and cell segregation of GBS as well as purine biosynthesis (Rajagopal L et al. J Biol. Chem. 2003). Homologues of stk1 have been identified in other streptococci species. stkP of S. pneumoniae had positive effect in competence phenotype (Echenique J et al. AIA 2004). In S. mutans, biofilm formation, competence and acid resistance required stkP gene (Hussain H et al. J. bact. 2006). Serine/threonine kinase had also a significant impact on the virulence of the streptococci in animal model (Echenique J et al. AIA 2004).

Analysis of gbs0100 gene product revealed the presence of a phosphomethylpyrimidine kinase motif and showed homology to thiD gene product found in many gram-positive bacteria such as B. subtilis (Park J H et al. J. bact. 2004). ThiD is evolved in the thiamine pyrophosphate (TPP) biosynthesis.

Gbs0653 was firstly identified as part of a genetic locus required for GBS β-hemolysin activity (Pritzlaff C A et al. Mol. mic. 2001). Protein, as member of the cyI operon, corresponded to CyIH in its N-terminus region and CyII constitute the C-terminal of the protein. Gbs0653 encode product with homology to ketoacyl-ACP synthase and had significant homology to fabF product of E. coli implicated in the fatty acid synthesis pathway. In S. agalactiae, expression of the cyI operon was shown to be tightly regulated by CovR/S two-component system (Lamy M C et al. Mol. mic. 2004)

Identification and Role of GBS Homologs in Gram-Positive Pathogen

Homology searching in publicly available microbial genome revealed that all genes found orthologues in the genome sequence of some relevant virulent gram-positive species. GBS genes, with the exception of gbs1787, matched in the genome of related Group A streptococci (S. pneumoniae and S. pyogenes) as well as in genome of some more distant species such as S. aureus, E. faecalis or B. anthracis. (Table 4). In almost all case, homology was not restricted to a small domain but covered the entire protein, which suggest that orthologous protein might be functional in other gram-positive bacteria and that this function might similar to that observed in S. agalactiae.

To test this hypothesis, gene knockout of paralogs of GBS virulence genes were performed in S. pneumoniae D39 by taking advantage of the natural competence of this strain. Phenotype of S. pneumoniae mutant was evaluated in a mice infection model. A determination of bacterial number was done in lung and blood of mice intravenously injected with S. pneumoniae D39. In contrast to GBS mutants, S. pneumoniae mutants displayed various phenotypes following mice inoculation. After intravenous injection, wild type D39 strain disseminated leading to bacteria and significant presence of bacteria in the lung. Deletion mutants such as sp1450 (gbs0100 homologs) and sp1979 (gbs1830) did not seem to be implicated in S. pneumoniae virulence as behaved as did the wild type strain. However, hypo-virulent phenotype was associated to mutants of genes sp1868 (gbs0052 homologs), sp1577 (gbs0307), sp1176 (gbs0582) and to a lesser extent sp2010 (gbs2100) by means of significant reduction in bacteraemia and lung dissemination (FIG. 2).

The invention relates also to the design of biochemical assays.

1; Biochemical assays as screening assays for inhibitors for target GBS 0307 is a bacterial Serine/threonine kinase catalysing the phosphorylation of diverse proteins, the nature of which is still in debate (an histone-like protein for S. pyogenes, an inorganic pyrophosphatase for S. agalactiae). GBS 0307 assays as described in the literature are essentially radioactivity-based (Journal of Molecular Biology, 2006, 357(5), p. 1351-1372 and Journal of Biological Chemistry, 2003, Vol. 278, 16, p. 14429-14441). The non-radioactive assays described below are based either on luminescent ATP detection, or on fluorescent ADP detection. They use the prototypical substrate MBP (myelin basic protein) and are easily amenable to miniaturized formats and fast readouts as required by HTS.

GBS 0307 Luminescent Assay

The assay buffer “AB” contains 50 mM Hepes pH7.5, 0.5 mM MnCl₂, 0.012% Triton-X100 and 1 mM DTT. The following components are added in a white polystyrene Costar plate up to a final volume of 30 μL: 3 μL DMSO, or inhibitor dissolved in DMSO and 27 μL MTB26 in AB. After 30 min of pre-incubation at room temperature, 30 μL of Substrates mix in AB are added in each well to a final volume of 60 μL. This reaction mixture is then composed of 5 nM GBS 0307 (produced in house from S. agalactiae), 0.3 μM myelin basic protein (Sigma) and 0.3 μM ATP (Sigma) in assay buffer. After 90 min of incubation at room temperature, 30 μL of the revelation mix are added to a final volume of 90 μL, including the following constituents at the respective final concentrations: 2 nM luciferase (Sigma), 30 μM D-luciferin (Sigma), 100 μM N-acetylcysteamine (Aldrich). Luminescence intensity is immediately measured on an Analyst-HT (Molecular Devices) and converted into inhibition percentages. For IC50 determinations, the inhibitor is tested at 6 to 10 different concentrations, and the related inhibitions are fitted to a classical langmuir equilibrium model using XLFIT (IDBS).

GBS 0307 Fluorescent Assay

The assay buffer “AB” contains 50 mM Hepes pH7.5, 0.5 mM MnCl₂, 0.012% Triton-X100 and 1 mM DTT. The following components are added in a black polystyrene Costar plate up to a final volume of 50 μL: 5 μL DMSO, or inhibitor dissolved in DMSO and 45 μL GBS 0307 in AB. After 30 min of pre-incubation at room temperature, 50 μL of Substrates-revelation mix in AB are added in each well to a final volume of 100 μL. This reaction mixture is then composed of 10 nM MTB26 (produced in house from S. agalactiae), 2 μM myelin basic protein (Sigma), 0.3 μM ATP (Sigma), 5 u/mL Pyruvate Kinase (Sigma), 50 μM phosphoenolpyruvate (Sigma), 5 u/mL Lactate deshydrogenase (Sigma) and 3 μM NADH (Sigma) in assay buffer. Fluorescence intensity of NADH (λₑₓ=360 nm, λₑₘ=520 nm) is immediately measured kinetically by a Fluostar Optima (BMG). Inhibition percentages are derived from fitted initial velocities. For IC50 determinations, the inhibitor is tested at 6 to 10 different concentrations, and the related inhibitions are fitted to a classical langmuir equilibrium model using XLFIT (IDBS).

Reference Inhibitor of GBS 0307

The inventors have shown that Staurosporine was inhibitor of MTB26 with an IC50 of 23±8 nM (FIG. 3).

2—Biochemical assays as screening assays for inhibitors for target GBS 0582

### INTRODUCTION

GBS 0582 is a protein of unknown function, comprising a DHH domain (related to a phosphoesterase-type activity) and a DHHA1 domain, presumably related to substrate recognition. From sequence comparison, it can be hypothesized that GBS 0582 hydrolyses a phosphoester bond of unknown nature (protein phosphatase, sugar phosphatase, pyro- or poly-phosphate hydrolase, RNAse, DNAse etc. . . . ). Any hydrolysis activity of MTB27 on pyro- nor poly-phosphates was not experimentally shown. But it was shown that 4-methylumbelliferylphosphate, an artificial substrate for many phosphatases, was recognized and hydrolysed by MTB27, thus generating a fluorescent signal through formation of the fluorophore 4-methylumbelliferone. As no literature exists in the field, this is the first report of an activity assay for GBS 0582, confirming its phosphoesterase function. The natural substrate has still to be discovered.

MTB27 Fluorescent Assay

The assay buffer “AB” contains 50 mM Hepes pH7.5, 20 mM MnCl₂, 0.006% Triton-X100, 2 mM DTT. The following components are added in a black polystyrene Costar plate up to a final volume of 60 μL: 3 μL DMSO, or inhibitor dissolved in DMSO, 47 μL 4-methylumbelliferylphosphate and 10 μL GBS 0582 in AB. This reaction mixture is composed of 10 nM GBS 0582 (produced in house from S. agalactiae) and 300 μM 4-methylumbelliferylphosphate (Sigma) in assay buffer. After 90 min of incubation, fluorescence intensity of 4-methylumbelliferone (λₑₓ=360 nm, λₑₘ=460 nm) is read on a Fluostar Optima (BMG) and converted into inhibition percentages. Alternatively, one can read the plate kinetically during the 90 min of incubation and derive inhibition percentages from fitted initial velocities. For IC50 determinations, the inhibitor is tested at 6 to 10 different concentrations, and the related inhibitions are fitted to a classical langmuir equilibrium model using XLFIT (IDBS).

Reference Inhibitor of GBS 0582

The inventors have shown that Adenosine monophosphate AMP was inhibitor of MTB27 with an IC50 of 235±45 μM. ADP inhibits MTB27 as well, but less efficiently (IC₅₀=405 μM) (FIG. 4)

Seq 1

>gi|23094478|emb|CAD45697.1| gbs0052 gene product
[Streptococcus agalactiae NEM316]
MRIKWFSLVRITGLLLVLLYHFFKNSFPGGFVGVDIFFTFSGFLITALLI
DEFSKTKKIDFVSFCRRRFYRIFPPLVLMVLVTIPFVFLVKSDFRASIGS
QIMTALGFTSNFYEILTGGNYESQFIPHLFVHTWSLSIEVHFYVLWGLTV
WLLSKRSKDQKQLRGTLFLISMGVFGVSFLTMFVRAFFVDNFSTIYFSTL
SHIFPFFLGAMVATISGIREITGRFKKNIKNLTLKHNLIMMGSAPAGLMI
LTFALDFDNRLTYLFGFVLSSIFASVMIYNARILHEHTPDISEPFVITYL
ADISYGMYLFHWPFYIIFSRLSPNWIAVILTVVLSAVFSTLSFYIIEPFI
LGRKPKFLDYEFDLLPYKKWLFSIGGVLTLITVVTMLTAPSIGSFETELL
QNSLQQARTNTNRTHTLAAGDAGALSDVTVIGDSVALRSSAAFNKLLPEV
QLDAAVSRNFSKSFDIFENRIQNKALSKIVVLAVGVNSLDNYKTDLSQFI
KSLPKGHRLIIVTPYNAKNMSQVTTVRDYELSLMKKYNYITVADWYKVAT
EHPEIWGNTDGVHYSDSDTTGADLYVSNVKKAIQKSAQRAAK

Seq 2

>gi|23094526|emb|CAD45745.1| gbs0100 gene product
[Streptococcus agalactiae NEM316]
MKTRNVLAISGNDIFSGGGLHADLATYVVNKLHGFVAVTCLTAMSDKGFE
VIPIEASILKQQLESLKDVEFGSIKLGLLPNVETAQVVLEFVKSKQECPV
VLDPVLVCKENHDLEVSQLREQLIAFFPYADVITPNLVEAQLLTGLSIEN
LDQMKIAAEKLYDMGAKHVVIKGGNRLRAEEATDLYYDGERFETYVFPVV
DANNTGAGCTFASSIASQLAMGENVEDAVKMSKGFVYQAIKASDKYGVVQ
HF

Seq 3

>gi|23094733|emb|CAD45952.1| gbs0307 gene product
[Streptococcus agalactiae NEM316]
MIQIGKLFAGRYRILKSIGRGGMADVYLARDLILDNEEVAIKVLRTNYQT
DQIAVARFQREARAMAELTHPNIVAIRDIGEEDGQQFLVMEYVDGFDLKK
YIQDNAPLSNNEVVRIMNEVLSAMSLAHQKGIVHRDLKPQNILLTKKGTV
KVTDFGIAVAFAETSLTQTNSMLGSVHYLSPEQARGSKATVQSDIYAMGI
MLFEMLTGHIPYDGDSAVTIALQHFQKPLPSILAENKSVPQALENIVIKA
TAKKLTDRYKTTYEMGRDLSTALSSTRHREPKLVFNDTESTKTLPKVTST
VSSLTTEQLLRNQKQAKTTEKITPDSASNDKTKSKKKASHRLLGTIMKLF
FALCVVGIIVFAYKILVSPTTIRVPDVSNKTVAQAKMTLENSGLKVGAIR
NIESDSVSEGLVVKTDPAAGRSRREGAKVNLYIATPNKSFTLGNYKEHNY
KDILKDLQGKGVKKSLIKVKRKINNDYTTGTILAQSLPEGTSFNPDGNKK
LTLTVAVNDPMIMPDVTGMTVGEVIETLTDLGLDADNLVFYQMQNGVYQA
VVTPPSSSKIASQDPYYGGEVGLRRGDKVKLYLLGSKTTNNSSSTPIDSS
ASSSTGTTTSDSVSSSTDASTSDSSSTSTSSSTLPSDSTTNTGTANNPLT
Q

Seq 4

>gi|23095005|emb|CAD46226.1| gbs0582 gene product
[Streptococcus agalactiae NEM316]
MIIFQQILDKIKEYDTIIIHRHMRPDPDALGSQIGLRDIIRHNFPKKKVL
ATGFDEPTLAWIAKMDQVTDQDYQGALVVVTDTANTPRIDDERYKKGDFL
IKIDHHPNDEVYGDLSYVDTNASSASEIVTDFALSCDLLLSTSAARVLYN
GIVGDTGRFLYPATTSKTLKIASKLREFDFDFSAMARQMDSFPFKIAKLQ
GFIFEQLKIDKNGAACVTLTQEDLKRFDVTDAETAAIVGVPGKIDIVESW
AIFVEQSDGHYRVRLRSKSHIINEIAKRHNGGGHPLASGANSYSLEENQA
IYQEIQEILSL

Seq 5

>gi|25010709|ref|NP_735104.1| gbs0653 gene product
[Streptococcus agalactiae NEM316]
MSVYVSGIGIISSLGKNYSEHKQHLFDLKEGISKHLYKNHDSILESYTGS
ITSDPEVPEQYKDETRNFKFAFTAFEEALASSGVNLKAYHNIAVCLGTSL
GGKSAGQNALYQFEEGERQVDASLLEKASVYHIADELMAYHDIVGASYVI
STACSASNNAVILGTQLLQDGDCDLAICGGCDELSDISLAGFTSLGAINT
EMACQPYSSGKGINLGEGAGFVVLVKDQSLAKYGKIIGGLITSDGYHITA
PKPTGEGAAQIAKQLVTQAGIDYSEIDYINGHGTGTQANDKMEKNMYGKF
FPTTTLISSTKGQTGHTLGAAGIIELINCLAAIEEQTVPATKNEIGIEGF
PENFVYHQKREYPIRNALNFSFAFGGNNSGILLSSLDSPLETLPARENLK
MAILSSVASISKNESLSITYEKVASNFNDFEALRFKGARPPKTVNPAQFR
KMDDFSKMVAVTTAQALIESNINLKKQDTSKVGIVFTTLSGPVEVVEGIE
KQITTEGYAHVSASRFPFTVMNAAAGMLSIIFKITGPLSVISTNSGALDG
IQYAKEMMRNDNLDYVILVSANQWTDMSFMWWQQLNYDSQMFVGSDYCSA
QVLSRQALDNSPIILGSKQLKYSHKTFTDVMTIFDAALQNLLSDLGLTIK
DIKGFVWNERKKAVSSDYDFLANLSEYYNMPNLASGQFGFSSNGAGEELD
YTVNESIEKGYYLVLSYSIFGGISFAIIEKR

Seg 6

>gi|23095092|emb|CAD46327.1| gbs0683 gene product
[Streptococcus agalactiae NEM316]
MRIGLFTDTYFPQVSGVSTSIRTLKEGLEKEGHEVYIFTTTDRNVKRFED
PTIIRLPSVPFISFTDRRVVYRGLISAYRIAKDYELDIIHTQTEFSLGLL
GKLVAKALRIPVVHTYHTQYEDYVGYIAKGKLIKPSMVKYIMRTYLSDLD
GVICPSRIVLNLLDGYGVKIPKQVIPTGIPVENYRREDISEETIKNLRTE
LGLADNDTMLLSLSRVSFEKNIQAILMHLSAVVDENPHVKLVIVGDGPYL
SDLKELVHSLELENSVIFTGMVEHSQVAIYYKACDFFISASTSETQGLTY
IESLASGRPIIAQSNPYLDDVISDKMFGTLYKKESDLADAILDAIAETPK
MTQEAYEQKLYEISAENFSKSVYAFYLDFLISQKASVKEKVSLTIGNKDS
HSTLRFVRKAVYLPKKVFTFTGRASKKVVKAPKRRISSIRDFLD

Seg 7

>gi|24413367|emb|CAD47446.1| gbs1787 gene product
[Streptococcus agalactiae NEM316]
MTIETLARFQFAMTTVFHFFFVPFTIGTCLVVAIMETMYVITKNEEYKKL
TKFWGNIMLLSFAVGVVTGIIQEFQFGMNWSDYSRFVGDIFGAPLAIEAL
LAFFMESTFLGLWMFTWDNKKISKKLHVTFIWLVVFGSLMSAMWILTANS
FMQHPVGYEVVNGRAQMTDFLALVKNPQFFYEFTHVIFGAITMGGTVVAG
MSAFRLLKSEQLKDTTVELYKKSVRIGLVVALLGSISVMGVGDLQMKALI
HDQPMKFAAMEGDYEDSGDPAAWSVVAWANEAEHKQVFGIKIPYMLSILS
YGKPSGSVKGMDTANKELVAKYGKDNYYPMVNLLFYGFRTMAAMGTAIMG
VSVLGLFLTRKKKPILYKHKWMLWIVALTTFAPFLANTFGWIVTEQGRYP
WTVYGLFKIKDSVSPNVSVASLFVSNTVYFLLFGGLAVMMISLTIRELKK
GPEYEDEHGHHGAYTSIDPFEKGAY

Seq 8

>gi|24413679|emb|CAD47759.1| gbs2100 gene product
[Streptococcus agalactiae NEM316]
MKRFRFATVHLVLIGLILFGLLAICVRLFQSYTALLLAIFVVLSFVVALL
YYQKITYELSEVEQIELLNDQTEVSLKSLLEQMPVGVIQFDLETNDIEWF
NPYAELIFTGDNGHFQSATVKDIITSRRNGTAGQSFEYGDNKYSAYLDTE
TGVFYFFDNFMGNRRNYDSSMLRPVIGIISIDNYDDIMDTMLEADMSKIN
AFVTSFISDFTQSKNIFYRRVNMDRYYIFTDYSVLNTLIKDKFDILNEFR
KRAQENHLSLTLSMGISYGDGNHNQIGQIALENLNTALVRGGDQIVVREN
DSSKKALYFGGGAVSTIKRSRTRTRAMMTAISDRLKVVDSVFIVGHRKLD
MDALGASVGMQFFASNIVNASYVVYDPNDMNSDIERAIDYLQEDGETRLV
SVERAFELITQNSLLVMVDHSKTALTLSKEFFNKFADVIVVDHHRRDEDF
PKNAVLSFIESGASSASELVTELIQFQQAKDKLSRSQASILMAGIMLDTR
NFASNVTSRTFDVASYLRGLGSNSMAIQKISATDFDEYRLINELILKGER
IYDNIIVATGEEHKVYSHVIASKAADTMLTMAGIEATFVITKNSSNIGIS
ARSRNNINVQRIMEKLGGGGHFSFAACQIQDKSVKQVRRMLLEIIDEDLR
ENSTVENRRD

1. A method for the insertion mutagenesis of GBS comprising the use of vector pTCV-Tase.

2. The insertion mutant of genes gbs 0052 (SEQ ID N° 1), gbs 0100 (SEQ ID N° 2), gbs 0307 (SEQ ID N° 3), gbs 0582 (SEQ ID N° 4), gbs 0653 (SEQ ID N° 5), gbs 0683 (SEQ ID N° 6), gbs 1787 (SEQ ID N° 7), gbs 2100 (SEQ ID N° 8).

3. In vitro Screening Method of the mutants library comprising using colistine as a mimic of innate immunity components that are the antibacterial cationic peptides and using novobiocine to detect mutant with defect in outer membrane permeability.

4. A methods combining the in vitro screening results with in vivo effect of mutations, to identify target proteins having en essential function for in vivo virulence.

5. Proteins sequences of the targets in GBS identified claim 1 as useful to find drugs preventing bacterial dissemination in the host.

6. The proteins of claim 1 which are in GBS and homologous sequences in gram positive bacteria with at least 22% identify on the full length seq and 25% identity in a 100 continuous amino acid sequence.

7. The proteins according to claim 6, wherein the homologous proteins are present in Streptococcus Pneumoniae (SPN), to find drugs for treating Gram positive infections.

8. A biochemical assay for screening inhibitors for GBS 0307
characterized in that they are based either on luminescent ATP or fluorescent ADP detection.

9. The biochemical assay of claim 8, comprising:
adding a substrate mixture comprising GBS, myelin basic protein and ATP to an assay buffer preincubated with DMSO or an analog or inhibitor dissolved in DMSO or analog,
incubating at room temperature,
adding a revelation mixture, and
measuring luminescence.

10. The biochemical assay of claim 6, comprising:
adding a substrate mixture comprising GBS, myelin basic protein; ATP, pyruvate and NaDH,
measuring fluorescence intensity of NaDH (λₑₓ=360 nm, λₑₘ=520 nm),
deriving the inhibition % from fitted initial velocities.


SEQUENCE LISTING (16 sequences)

SEQ ID NO: 1 - protein, 592 aa, Streptococcus agalactiae
MRIKWFSLVRITGLLLVLLYHFFKNSFPGGFVGVDIFFTFSGFLITALLIDEFSKTKKIDFVSFCRRRFYRIFPPLVLMVLVTIPFVFLVKSDFRASIGSQIMTALGFTSNFYEILTGGNYESQFIPHLFVHTWSLSIEVHFYVLWGLTVWLLSKRSKDQKQLRGTLFLISMGVFGVSFLTMFVRAFFVDNFSTIYFSTLSHIFPFFLGAMVATISGIREITGRFKKNIKNLTLKHNLIMMGSAFAGLMILTFALDFDNRLTYLFGFVLSSIFASVMIYNARILHEHTPDISEPFVITYLADISYGMYLFHWPFYIIFSRLSPNWIAVILTVVLSAVFSTLSFYIIEPFILGRKPKFLDYEFDLLPYKKWLFSIGGVLTLITVVTMLTAPSIGSFETELLQNSLQQARTNTNRTHTLAAGDAGALSDVTVIGDSVALRSSAAFNKLLPEVQLDAAVSRNFSKSFDIFENRIQNKALSKIVVLAVGVNSLDNYKTDLSQFIKSLPKGHRLIIVTPYNAKNMSQVTTVRDYELSLMKKYNYITVADWYKVATEHPEIWGNTDGVHYSDSDTTGADLYVSNVKKAIQKSAQRAAK

SEQ ID NO: 2 - protein, 252 aa, Streptococcus agalactiae
MKTRNVLAISGNDIFSGGGLHADLATYVVNKLHGFVAVTCLTAMSDKGFEVIPIEASILKQQLESLKDVEFGSIKLGLLPNVETAQVVLEFVKSKQECPVVLDPVLVCKENHDLEVSQLREQLIAFFPYADVITPNLVEAQLLTGLSIENLDQMKIAAEKLYDMGAKHVVIKGGNRLNAEEATDLYYDGERFETYVFPVVDANNTGAGCTFASSIASQLAMGKNVEDAVKMSKGFVYQAIKASDKYGVVQHF

SEQ ID NO: 3 - protein, 651 aa, Streptococcus agalactiae
MIQIGKLFAGRYRILKSIGRGGMADVYLARDLILDNEEVAIKVLRTNYQTDQIAVARFQREARAMAELTHPNIVAIRDIGEEDGQQFLVMEYVDGFDLKKYIQDNAPLSNNEVVRIMNEVLSAMSLAHQKGIVHRDLKPQNILLTKKGTVKVTDFGIAVAFAETSLTQTNSMLGSVHYLSPEQARGSKATVQSDIYAMGIMLFEMLTGHIPYDGDSAVTIALQHFQKPLPSILAENKSVPQALENIVIKATAKKLTDRYKTTYEMGRDLSTALSSTRHREPKLVFNDTESTKTLPKVTSTVSSLTTEQLLRNQKQAKTTEKITPDSASNDKTKSKKKASHRLLGTIMKLFFALCVVGIIVFAYKILVSPTTIRVPDVSNKTVAQAKMTLENSGLKVGAIRNIESDSVSEGLVVKTDPAAGRSRREGAKVNLYIATPNKSFTLGNYKEHNYKDILKDLQGKGVKKSLIKVKRKINNDYTTGTILAQSLPEGTSFNPDGNKKLTLTVAVNDPMIMPDVTGMTVGEVIETLTDLGLDADNLVFYQMQNGVYQAVVTPPSSSKIASQDPYYGGEVGLRRGDKVKLYLLGSKTTNNSSSTPIDSSASSSTGTTTSDSVSSSTDASTSDSSSTSTSSSTLPSDSTTNTGTANNPLTQ

SEQ ID NO: 4 - protein, 311 aa, Streptococcus agalactiae
MIIFQQILDKIKEYDTIIIHRHMRPDPDALGSQIGLRDIIRHNFPKKKVLATGFDEPTLAWIAKMDQVTDQDYQGALVVVTDTANTPRIDDERYKKGDFLIKIDHHPNDEVYGDLSYVDTNASSASEIVTDFALSCDLLLSTSAARVLYNGIVGDTGRFLYPATTSKTLKIASKLREFDFDFSAMARQMDSFPFKIAKLQGFIFEQLKIDKNGAACVTLTQEDLKRFDVTDAETAAIVGVPGKIDIVESWAIFVEQSDGHYRVRLRSKSHIINEIAKRHNGGGHPLASGANSYSLEENQAIYQEIQEILSL

SEQ ID NO: 5 - protein, 731 aa, Streptococcus agalactiae
MSVYVSGIGIISSLGKNYSEHKQHLFDLKEGISKHLYKNHDSILESYTGSITSDPEVPEQYKDETRNFKFAFTAFEEALASSGVNLKAYHNIAVCLGTSLGGKSAGQNALYQFEEGERQVDASLLEKASVYHIADELMAYHDIVGASYVISTACSASNNAVILGTQLLQDGDCDLAICGGCDELSDISLAGFTSLGAINTEMACQPYSSGKGINLGEGAGFVVLVKDQSLAKYGKIIGGLITSDGYHITAPKPTGEGAAQIAKQLVTQAGIDYSEIDYINGHGTGTQANDKMEKNMYGKFFPTTTLISSTKGQTGHTLGAAGIIELINCLAAIEEQTVPATKNEIGIEGFPENFVYHQKREYPIRNALNFSFAFGGNNSGILLSSLDSPLETLPARENLKMAILSSVASISKNESLSITYEKVASNFNDFEALRFKGARPPKTVNPAQFRKMDDFSKMVAVTTAQALIESNINLKKQDTSKVGIVFTTLSGPVEVVEGIEKQITTEGYAHVSASRFPFTVMNAAAGMLSIIFKITGPLSVISTNSGALDGIQYAKEMMRNDNLDYVILVSANQWTDMSFMWWQQLNYDSQMFVGSDYCSAQVLSRQALDNSPIILGSKQLKYSHKTFTDVMTIFDAALQNLLSDLGLTIKDIKGFVWNERKKAVSSDYDFLANLSEYYNMPNLASGQFGFSSNGAGEELDYTVNESIEKGYYLVLSYSIFGGISFAIIEKR

SEQ ID NO: 6 - protein, 444 aa, Streptococcus agalactiae
MRIGLFTDTYFPQVSGVSTSIRTLKEGLEKEGHEVYIFTTTDRNVKRFEDPTIIRLPSVPFISFTDRRVVYRGLISAYRIAKDYELDIIHTQTEFSLGLLGKLVAKALRIPVVHTYHTQYEDYVGYIAKGKLIKPSMVKYIMRTYLSDLDGVICPSRIVLNLLDGYGVKIPKQVIPTGIPVENYRREDISEETIKNLRTELGLADNDTMLLSLSRVSFEKNIQAILMHLSAVVDENPHVKLVIVGDGPYLSDLKELVHSLELENSVIFTGMVEHSQVAIYYKACDFFISASTSETQGLTYIESLASGRPIIAQSNPYLDDVISDKMFGTLYKKESDLADAILDAIAETPKMTQEAYEQKLYEISAENFSKSVYAFYLDFLISQKASVKEKVSLTIGNKDSHSTLRFVRKAVYLPKKVFTFTGRASKKVVKAPKRRISSIRDFLD

SEQ ID NO: 7 - protein, 475 aa, Streptococcus agalactiae
MTIETLARFQFAMTTVFHFFFVPFTIGTCLVVAIMETMYVITKNEEYKKLTKFWGNIMLLSFAVGVVTGIIQEFQFGMNWSDYSRFVGDIFGAPLAIEALLAFFMESTFLGLWMFTWDNKKISKKLHVTFIWLVVFGSLMSAMWILTANSFMQHPVGYEVVNGRAQMTDFLALVKNPQFFYEFTHVIFGAITMGGTVVAGMSAFRLLKSEQLKDTTVELYKKSVRIGLVVALLGSISVMGVGDLQMKALIHDQPMKFAAMEGDYEDSGDPAAWSVVAWANEAEHKQVFGIKIPYMLSILSYGKPSGSVKGMDTANKELVAKYGKDNYYPMVNLLFYGFRTMAAMGTAIMGVSVLGLFLTRKKKPILYKHKWMLWIVALTTFAPFLANTFGWIVTEQGRYPWTVYGLFKIKDSVSPNVSVASLFVSNTVYFLLFGGLAVMMISLTIRELKKGPEYEDEHGHHGAYTSIDPFEKGAY

SEQ ID NO: 8 - protein, 660 aa, Streptococcus agalactiae
MKRFRFATVHLVLIGLILFGLLAICVRLFQSYTALLLAIFVVLSFVVALLYYQKITYELSEVEQIELLNDQTEVSLKSLLEQMPVGVIQFDLETNDIEWFNPYAELIFTGDNGHFQSATVKDIITSRRNGTAGQSFEYGDNKYSAYLDTETGVFYFFDNFMGNRRNYDSSMLRPVIGIISIDNYDDIMDTMLEADMSKINAFVTSFISDFTQSKNIFYRRVNMDRYYIFTDYSVLNTLIKDKFDILNEFRKRAQENHLSLTLSMGISYGDGNHNQIGQIALENLNTALVRGGDQIVVRENDSSKKALYFGGGAVSTIKRSRTRTRAMMTAISDRLKVVDSVFIVGHRKLDMDALGASVGMQFFASNIVNASYVVYDPNDMNSDIERAIDYLQEDGETRLVSVERAFELITQNSLLVMVDHSKTALTLSKEFFNKFADVIVVDHHRRDEDFPKNAVLSFIESGASSASELVTELIQFQQAKDKLSRSQASILMAGIMLDTRNFASNVTSRTFDVASYLRGLGSNSMAIQKISATDFDEYRLINELILKGERIYDNIIVATGEEHKVYSHVIASKAADTMLTMAGIEATFVITKNSSNIGISARSRNNINVQRIMEKLGGGGHFSFAACQIQDKSVKQVRRMLLEIIDEDLRENSTVENRRD

SEQ ID NO: 9 - DNA, 26 bp, Artificial sequences
  Oligonucleotides
cctatcacctcaaatggttcgctggg

SEQ ID NO: 10 - DNA, 24 bp, Artificial sequences
  Oligonucleotides
ctggggatcaagcctgattgggag

SEQ ID NO: 11 - DNA, 32 bp, Artificial sequences
  Oligonucleotides
atatccatggatggaaaaaaaggaatttcgtg

SEQ ID NO: 12 - DNA, 33 bp, Artificial sequences
  Oligonucleotides
aatctgcagttattattcaacatagttcccttc

SEQ ID NO: 13 - DNA, 25 bp, Artificial sequences
  Primers
tagcttattcctccaaggcacgagc

SEQ ID NO: 14 - DNA, 15 bp, Artificial sequences
  Primers
gatcgctcgtgcctt

SEQ ID NO: 15 - DNA, 24 bp, Artificial sequences
  Primers
cgctcttgaagggaactatgttga

SEQ ID NO: 16 - DNA, 24 bp, Artificial sequences
  Primers
aatcatttgaaggttggtactata